(19) United States
(12) Patent Application Publication
(10) Pub. No.: US 2024/0207476 A1
(43) Pub. Date: Jun. 27, 2024

(54) INTERCONNECTED ROOM TREATMENT

(71) Applicant: Scott A. Elrod, Lake Jackson, TX (US)

(72) Inventor: Scott A. Elrod, Lake Jackson, TX (US)

(21) Appl. No.: 18/146,578

(22) Filed: Dec. 27, 2022

(51) Int. Cl. A61L 2/24; A61L 2/10; A61L 2/20; A61L 9/22

(52) U.S. Cl. CPC A61L 2/24; A61L 2/10; A61L 2/202; A61L 9/22; A61L 2202/14; A61L 2202/25; A61L 2209/111; A61L 2209/14

(57) ABSTRACT

Embodiments of the invention relate to room treatment devices, systems, and methods for sanitizing rooms and communicating emergency conditions in a wireless network formed between the room treatment devices.

### BACKGROUND

Room sanitization is important to stop or slow the spread of illnesses in high traffic or communal areas. Room sanitization typically includes washing, scrubbing, mopping, or spraying surfaces with disinfectants. Further, to ameliorate smells, people often spray air fresheners and clean up the source of the smell. Such sanitization is typically carried out by people, such as janitorial staff, which can be costly and time consuming. This can be especially true in buildings available to the public, such as in hotels, schools, hospitals, churches, malls, and the like.

Safety concerns over bad actors in enclosed spaces, such as hospitals, hotels, schools, churches, malls, places of employment, and the like, are rising. Typically, an alarm is not raised until the bad actor harms someone. In many cases, the alarm may be the sound of gunfire, screaming, people running, or the like.

### SUMMARY

Embodiments of the invention relate to room treatment devices, systems, and methods for sanitizing rooms and communicating emergency conditions in a wireless network formed between the room treatment devices.

In an embodiment, a room treatment device having networking capabilities is disclosed. The room treatment device includes a room treatment source. The room treatment device includes a controller configured to control treatment output from the room treatment source. The room treatment device includes a wireless communication hub configured to receive and propagate one or more wireless signals from other room treatment devices.

In an embodiment, a system for treating a plurality of enclosed spaces is disclosed. The system includes a plurality of room treatment devices, each room treatment device of the plurality of room treatment devices including a room treatment source, a controller configured to control treatment output from the room treatment source, and a wireless communication hub configured to receive and propagate one or more wireless signals from other room treatment devices of the plurality of room treatment devices.

In an embodiment, a method for treating a room is disclosed. The method includes emitting a room treatment from a room treatment device, the room treatment device including a room treatment source, a controller configured to control treatment output from the room treatment source, and a wireless communication hub configured to receive and propagate one or more wireless signals from additional room treatment devices. The method includes maintaining a wireless connection with the additional room treatment devices.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the invention, wherein identical reference numerals refer to identical or similar elements or features in different views or embodiments shown in the drawings.

FIG. 1 is a block diagram of a room treatment device having networking capabilities, according to an embodiment.

FIG. 2 is an isometric view of the room treatment device, according to an embodiment.

FIG. 3 illustrates a use case of the room treatment device, according to an embodiment

FIG. 4 is a system for treating a plurality of enclosed spaces, according to an embodiment.

FIG. 5 is a flow diagram of a method for treating a room, according to an embodiment.

### DETAILED DESCRIPTION

Embodiments of the invention relate to devices, systems, and methods for treating rooms to remove scents, sterilize, clean or otherwise treat rooms using room treatment devices with device-to-device wireless connectivity. Due to bacteria, viruses, fungi, and other contaminants that spread, high traffic and communal areas need to be sanitized often in order to prevent the spread of illnesses an illness causing substrates. For example, schools and classrooms are often cited as a breeding ground for illnesses in communities. By providing a room treatment device in rooms, the room may be treated when unoccupied. The room treatment devices, systems, and methods disclosed herein can also provide device-to-device wireless communication between the room treatment devices in separate rooms to propagate wireless signals between the devices. Accordingly, the devices, systems, and methods disclosed herein allow users to provide alarm signals between devices and rooms to inform users in other rooms about concerning events, such as fire, medical emergencies, attackers (e.g., school or hospital shooters), fights between students, or the like.

The room treatment devices disclosed herein include at least a room treatment source (for example and without limitation, an ozone generator, a scent emitter, an ultraviolet light source, etc.), a controller configured to control treatment output from the room treatment source, and a wireless communication hub configured to receive and propagate one or more wireless signals from other room treatment devices in communication with the room treatment device. Systems of multiple room treatment devices in multiple rooms provide early warning to persons in other rooms from the room in which the room treatment device upon which the alarm signal is initiated.

In the systems disclosed herein, only one room treatment device of a plurality of room treatment devices may be in range of an external wireless network (e.g., Wi-Fi) or connected to a local area network (LAN). The connection to the external wireless network may be utilized to communicate the status of, alarm states of, or recordings from the room treatment devices to remote entities, such as emergency personnel (e.g., police, emergency medical services, fire department).

The methods disclosed herein utilize the room treatment devices to treat rooms and use the device-to-device wireless communication to communicate emergency situations between room treatment devices and rooms even in the absence of a Wi-Fi connection on every room treatment device. The room treatment devices may be controlled using a remote control or directly from the controls on the room treatment device. It will be understood by persons skilled in the art that some or all of the necessary and/or basic electronic equipment required to establish the room-to-room treatment devices, the communication hub, and any other electronic element for the wireless network may be hardwired, as needed or desired.

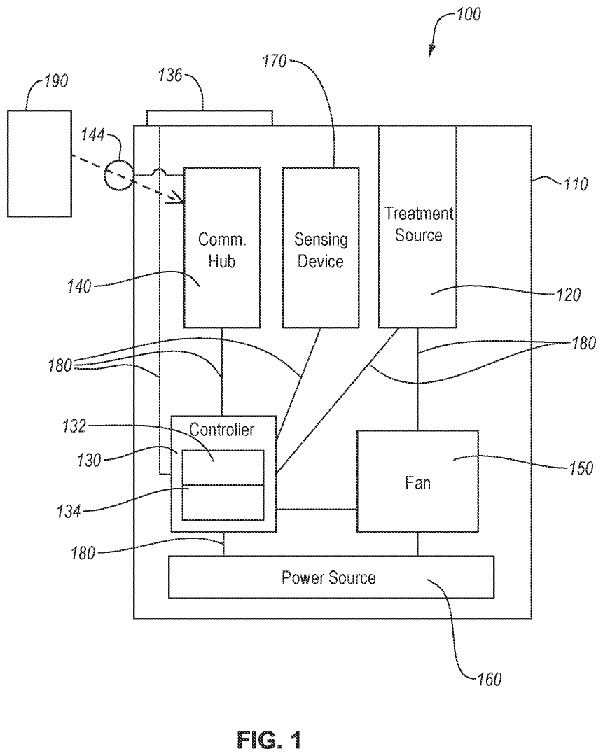

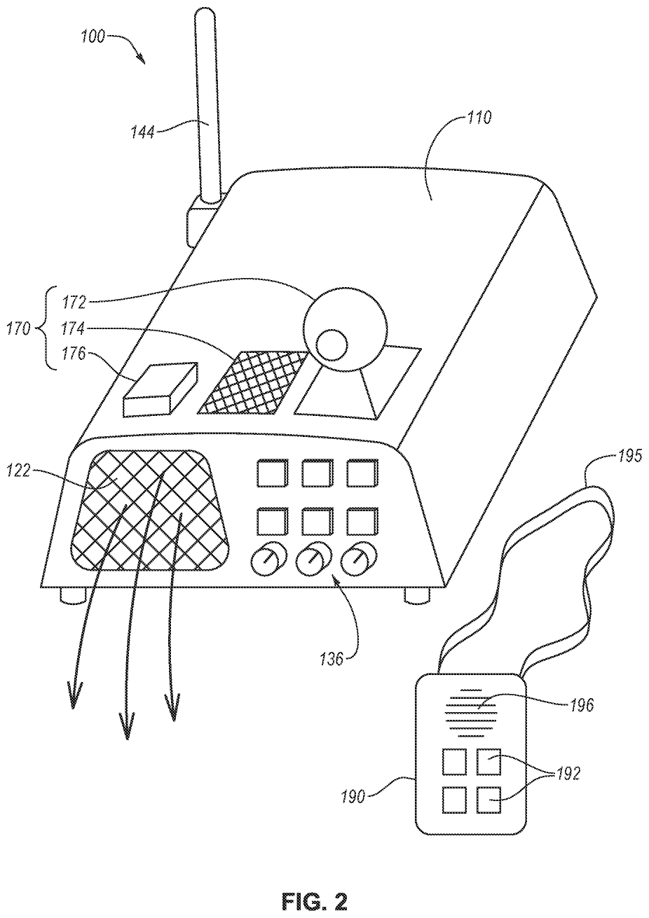

FIG. 1 is a block diagram of a room treatment device 100 having networking capabilities, according to an embodiment. FIG. 2 is an isometric view of the room treatment device 100, according to an embodiment. The room treatment device 100 includes a housing 110, a room treatment source 120, a controller 130 configured to control treatment output from the room treatment source 120, and a wireless communication hub 140 configured to receive and propagate one or more wireless signals from other room treatment devices, one or more fans 150, and a power source 160. The room treatment device 100 may include one or more sensing devices 170. The components of the room treatment device 100 are electrically connected via circuitry 180. A plurality of room treatment devices may be interconnected to create room-to-room connectivity. The room treatment device 100 may include a remote control to control one or more operations thereof.

The housing 110 includes a framework for storing components of the room treatment device 100. The housing 110 is sized and shaped to contain the components of room treatment device 100. For example, the room treatment source 120, the controller 130, the wireless communication hub 140, the fan 150, the power source 160, the one or more sensing devices 170, and the circuitry 180 may be at least partially disposed within or on the housing 110. The housing 110 may be constructed of one or more of metals, polymers, composites (e.g., carbon fiber), ceramics, or the like.

The housing 110 may include an outlet 122 for the treatment to pass through. For example, the outlet 122 may be a grate, screen, or other body for a room treatment material (e.g., ozone, ions, scent material) to pass through. In some examples, the housing 110 may include a plurality of outlets 122 such as one or more of an intake grate, a cooling grate, or other grates (not shown). For example, an intake grate may be located on a back, bottom, or top surface of the housing 110 to draw air into the fan 150 and/or the room treatment source 120.

The housing 110 may include legs for creating a space between the housing and a surface upon which the room treatment device 100 is mounted. In some examples, the housing may include a mounting bracket, fixture, or structure for mounting the room treatment device 100 on a wall or ceiling.

The room treatment source 120 is configured to provide a treatment to a room, such as to clean, descent, or otherwise sanitize the air, one or more objects, or one or more surfaces in a room or other discrete area. For example, the room treatment device may include one or more of an ozone generator, an ion generator, an ultraviolet light source, a scent source, an anti-microbial source, a humidifier, a dehumidifier, or an air filter. Accordingly, the room treatment source 120 may be configured to output one or more of ozone, hydroxide ions, hydride ions, a selected scent, ultraviolet light, or anti-microbial material.

Suitable ozone generators may include a corona discharge generator, an ultraviolet ozone generator, an electrolytic ozone generator, or the like. Suitable ion generators may include a negative ion generator, a bipolar ion generator, an electrostatic discharge ionizer, or the like. Suitable ultraviolet light sources may include a full spectrum UV light source, a UV-C light source, or another UV light source, such as a bulb or light emitting diode. Suitable scent sources may include a scent diffuser (e.g., evaporating aroma diffuser, piezoelectric diffuser, wick diffuser, or heated diffuser), a sprayer (e.g., an aerosol or mist dispenser), a gravity drip diffuser, a nebulizer, an atomizer, or the like. Suitable anti-microbial sources may include a sprayer, a diffuser, an atomizer or the like. Suitable a humidifiers may include a cool mist humidifier, a warm mist humidifier, an ultrasonic humidifier, an evaporative humidifier, or a vaporizer. Suitable dehumidifiers may include a refrigerant dehumidifier or a desiccant dehumidifier. The room treatment source 120 may include an air filter, such as an electrostatic air, a HEPA filter, a UV light filter, a media filter, a pleated filter, a spun glass air filter, a washable filter, or the like.

Particularly effective room treatment sources include ozone sources, such as corona discharge generators or even ion generators. Such room treatment sources provide for fast and effective treatment of a room.

Control of the room treatment source 120 may carried out by the controller 130. For example, the controller 130 is configured to control treatment output from the room treatment source 120. The controller 130 includes a memory storage medium 132 storing one or more operational programs thereon, the one or more operational programs including machine readable and executable instructions for treatment output from the room treatment source. The controller 130 includes a processor 134 operably coupled to the memory storage medium 132 and configured to execute the one or more operational programs. For example, the processor 134 may access, read, and execute the one or more operational programs to control output of the room treatment from the room treatment source, communicate with additional room treatment devices via the wireless communication hub (e.g. , transmit alarm signals), record sounds or videos with the sensing devices 170, or automatically control any other aspect of operation of the room treatment device 100.

The one or more operational programs may include machine readable and executable instructions for outputting one or more of ozone, hydroxide ions, hydride ions, a selected scent, ultraviolet light, or anti-microbial material according to a schedule. The one or more operational programs may include machine readable and executable instructions for producing an alarm responsive to an alarm signal received by the wireless communication hub, such as from an additional room treatment device. The one or more operational programs may include machine readable and executable instructions for propagating the alarm signal to all additional room treatment devices in communication with the wireless communication hub responsive to the alarm signal.

The operational programs may include machine readable and executable instructions for determining if the alarm signal is a unique alarm signal (e.g., has not been previously received from an additional room treatment device) so that a specific alarm signal corresponding to a specific event is not used or propagated multiple times. For example, a unique alarm signal identifier may be produced from the room treatment device where the alarm signal is initiated. Upon receiving the alarm signal with the unique alarm signal identifier from the initial room treatment device, an additional room treatment device may determine if the alarm signal with the unique alarm signal identifier has been received from any other room treatment device than the initial room treatment device. If the additional room treatment device has not previously received the signal from any other room treatment device (and produced the alarm corresponding thereto), instructions direct the additional room treatment device to sound an alarm and propagate the alarm signal to further additional room treatment devices in wireless communication therewith. If the additional room treatment device has previously received the signal from another room treatment device (and produced the alarm corresponding thereto), the instructions direct the additional room treatment device to refrain from sounding an alarm and/or propagating the alarm signal further.

The room treatment device 100 may include controls 136 (e.g., manual inputs) disposed on the housing 110 or outer surface thereof. The controls 136 are operably coupled to the controller 130 to communicate manual inputs thereto. The controls 136 may include inputs for amount, duration, type, etc. of room treatment. The controls 136 may include inputs for controlling a volume of a speaker on the room treatment device 100. The controls 136 may include inputs for initiating an alarm signal. The controls 136 may include inputs for initiating a recording. The controls 136 may include inputs for initiating a wireless connection with one or more additional room treatment devices or an external wireless connection (e.g., Wi-Fi) from a source other than the room treatment devices.

The one or more operational programs may include machine readable and executable instructions for initiating an alarm and propagating an alarm signal via the wireless communication hub 140 responsive to input from the remote control 190 or manual input on the room treatment device 100. For example and as explained in more detail below, the machine readable and executable instructions may include instructions for initiating an alarm signal responsive to a selected number of button pushes, a panic button push, or a selected code being entered.

Signals between the remote control 190 and the controller 130, between additional room treatment devices and the room treatment device 100, or a wireless network (e.g., Wi-Fi network) not formed by the room treatment devices are received and sent through the wireless communication hub 140. For example, the wireless communication hub 140 is configured to receive and propagate one or more wireless signals from other room treatment devices. The wireless communication hub 140 may include one or more of a router, transceiver, Bluetooth transmitter and receiver, infrared transmitter and receiver, or the like.

As shown in FIG. 2, the wireless communication hub 140 may include one or more antennas 144 for receiving and transmitting wireless signals. The antenna 144 may be an external antenna or an internal antenna (e.g., contained within the housing 110). Wireless signals are received by the antenna, processed in the wireless communication hub 140, and may be communicated to the controller 130.

The wireless communication hub 140 may be configured to receive, decode (e.g., read), and transmit wireless signals such as one or more of Bluetooth, Wi-Fi, cellular, infrared, radio signals, or the like. The wireless communication hub 140 is equipped and programmed to wirelessly connect to additional room treatment devices within a wireless communication range of the room treatment device 100. Accordingly, the wireless communication hub 140 may daisy chain a wireless signal to additional room treatment devices, thereby forming a discrete network capable of transmitting wireless signals in locations without a Wi-Fi connection from another source.

The room treatment device may include one or more fans 150. For example, the one or more fans 150 may include a fan 150 for passing ambient air through the treatment source (e.g., over corona discharge coils), through a filter, over the controller or communication hub for cooling, or outputting the room treatment (e.g., propelling ozone, ions, scent material or the like from the housing 110 or treatment source 120). Accordingly, the one or more fans 150 may include a blower, a cooling fan, an intake fan, or combinations of any of the foregoing.

The room treatment device 100 includes a power source 160. The power source 160 may include a power cord, such as a plug-in connection (e.g., three prong 110 volt North American connection, two prong European connection, etc.) for supplying power to the components of the room treatment device 100. The power source 160 may include one or more batteries, such as rechargeable batteries or capacitors. For example, the batteries may be replaceable batteries.

The room treatment device 100 may optionally include one or more sensing devices 170. The one or more sensing devices 170 may include an audio receiver or transceiver 174 (e.g., microphone), a video camera 172, a motion detector 176, a thermometer (not shown), a humidity sensor (not shown), or the like. The one or more sensing devices may include a speaker (shown as transceiver 174), such as a speaker for providing an audible alarm in the room in which the room treatment device is disposed. The speaker may be used to communicate verbal messages via the room treatment devices, such as from room treatment device to room treatment device in different rooms.

Referring back to the memory storage medium 132, the one or more operational programs may include instructions for emitting an alarm responsive to receiving an alarm signal from an additional room treatment device, manual input to initiate an alarm (e.g., from the controls 136), or remote input (e.g., from the remote control 190) to initiate an alarm, from the speaker. For example, responsive to a selected number of button pushes or a selected sequence of button pushes (e.g., a code) the operational program may include instructions to initiate an alarm and provide an alarm signal to additional room treatment devices in communication therewith. The one or more operational programs may include instructions for initiating an alarm and providing an alarm signal to additional room treatment devices in communication therewith responsive to a manual input, remote input, or sound detected above a threshold level (e.g., indicative of a fight, gunshot, or the like), or identifying a firearm in images.

The one or more operational programs may include instructions for ceasing treatment output if a sound is detected by the microphone or motion is detected by the motion detector or the video camera. The one or more operational programs may include instructions for recording one or more of sound via the microphone or video on the video camera, such as responsive to a manual input, remote input, sound detected above a threshold level, or identifying a firearm in images. The one or more operational programs may include instructions for wirelessly transmitting the one or more of the sound or video to an administrator, emergency services, or a remote storage site, such as a server.

The circuitry 180 includes circuitry connecting the components of the room treatment device 100, such as wires, circuits, circuit boards, or the like. The circuitry 180 includes power supply circuitry and data communication circuitry.

The room treatment device 100 may include remote control 190. The remote control 190 is in wireless communication with one or more of the controller 130 or the wireless communication hub 140. The remote control 190 is configured to control one or more functions or operations of the room treatment device 100. For example, the remote control 190 may include buttons 192 or a receiver (e.g., microphone or transceiver) or speaker for controlling one or more aspects of the room treatment device 100. The one or more of buttons or receiver are configured to send one or more signals to the controller 130, such as based on button pushes or verbal commands to actuate one or more functions of the room treatment device. The one or more functions may include initiating an alarm, initiating an alarm signal, propagating an alarm signal, initiating room treatment, adjusting room treatment parameters (e.g., duration, amount, type), terminating room treatment parameters, initiating a recording, sending a recording to a storage device, communicating an audio message via the room treatment devices, or any other functionality of the room treatment device disclosed herein.

Referring back to memory storage medium 132, the one or more operational programs may include machine readable and executable instructions for receiving inputs from the remote control 190 and determining which functions the inputs indicate should be executed. For example, the one or more operational programs include machine readable and executable instructions for initiating an alarm and propagating an alarm signal via the wireless communication hub responsive to input from the remote control or manual input on the room treatment device. According to some operational programs, the number of button pushes may indicate that an alarm should be initiated and an alarm signal should be sent to additional room treatment devices (indicative of a school shooter, hospital shooter, or the like) or that a recording should be initiated. According to some operational programs, a selected sequence of button pushes may indicate that an alarm should be initiated and an alarm signal should be sent to additional room treatment devices or that a recording should be initiated. In some embodiments, different numbers or sequences of button pushes may provide different commands (e.g., operate different functions of the room treatment device). For example, one button push may activate a mode for receiving inputs, two button pushes may send a signal indicating that non-emergency help is needed, three button pushes may send a signal indicating that a fire is present (and sound an alarm), four button pushes may indicate a shooter is present (and sound an alarm). Any of the functionalities disclosed herein may be associated with a selected number of button pushes or sequence of button pushes. In some examples, a panic button may be utilized to initiate an alarm signal.

One or more verbal inputs, such as a code word or phrase, may command, initiate, terminate or otherwise operate any of the functionalities disclosed herein, such as a room treatment should be initiated or terminated, a non-emergency alarm signal should be sent, or an alarm signal should be sent (e.g., emergency alarm signal).

The remote control 190 may be disposed on a lanyard 195, for wearing the remote control 190 around the neck of a user. In some embodiments, the remote control 190 may include a belt clip, hanger, or the like. In some examples the remote control 190 may be configured as a watch or otherwise wearable on a wrist or arm of the user.

In some embodiments, the remote control 190 may include software and hardware for determining and transmitting a position of the remote control 190, such as to the corresponding room treatment device or to a computing device or network. For example, the remote control 190 may include a global positioning satellite receiver or the like to identify the location the remote control. In such embodiments, the remote control 190 may inform users of the location of the remote control 190, such as to selectively locate the position of an emergency situation or the wearer of the remote control 190. The position of the remote control 190 may be actively or passively monitored by the room treatment device, such as via the antenna 144 and the communications hub. For example, the remote control 190 may periodically provide a position signal to the room treatment device or may provide a position signal upon activating an alarm signal or a location function on the remote control 190 or room treatment device. Such location software and hardware allow the room treatment devices and systems to identify the location of an alarm initiation. In some embodiments, the remote control may include software and hardware for emitting an audible alarm to audibly indicate the location of the remote control 190.

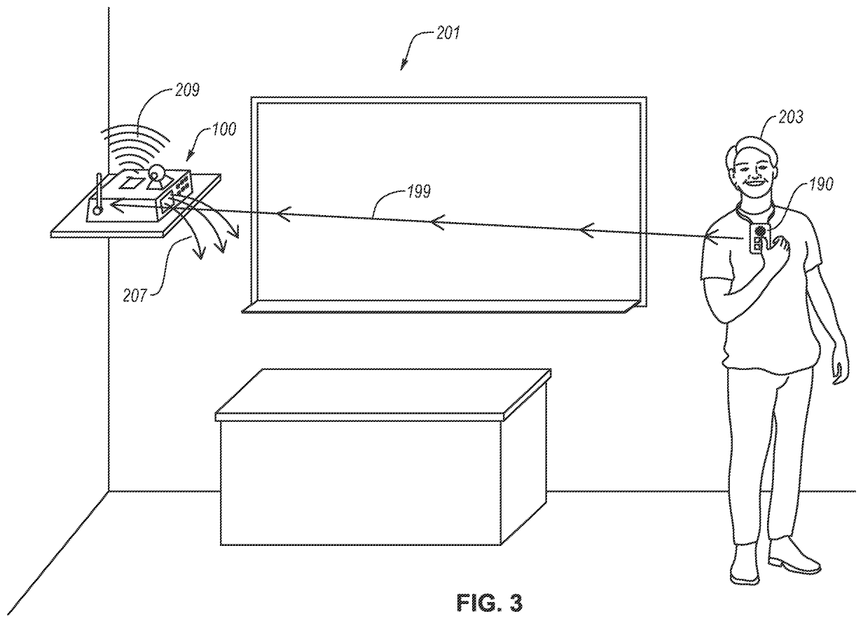

FIG. 3 is illustrates a use case of the room treatment device 100, according to an embodiment. The room treatment device 100 may be disposed in a classroom 201 to treat the room 201 with a room treatment 207. The room treatment may be an ozone treatment to treat an odor in the room or to sanitize the room prior to using the room. For example, the room treatment device 100 may include a corona discharge ozone generator for generating ozone and treating rooms with ozone to sanitize the rooms and objects therein. Upon or prior to entering the room, a user 203 may use the remote control 190 to terminate output of the room treatment 207. Likewise, upon leaving the room, the user may initiate output of the room treatment 207. The room treatment 207 may be run according to an operational program that indicates a set duration, output rate, and type of treatment. The wireless communication 199 between the remote control 190 and the room treatment device 100 is depicted as one-way but may include two way communication, such as alerts or messages relayed from additional room treatment devices or status identifiers of the room treatment device 100 (e.g. , operational status, run time left on a room treatment, etc.).

The wireless communication 199 may include inputs to raise an alarm and initiate an alarm signal. Upon activation of an alarm via the wireless communication 199, an audible alarm 209 may my emitted from the room treatment device 100. The wireless communication 199 may include inputs to initiate and/or transmit a recording, such as a sound and/or video recording for liability or criminal prosecution purposes.

While a classroom is depicted, the room treatment devices, systems, and methods disclosed herein are not limited to use in schools. For example, the room treatment devices, systems, and methods disclosed herein may be used in hospitals, hotels, churches, office buildings, conference centers, cruise ships, court houses, police stations, places or employment, or the like.

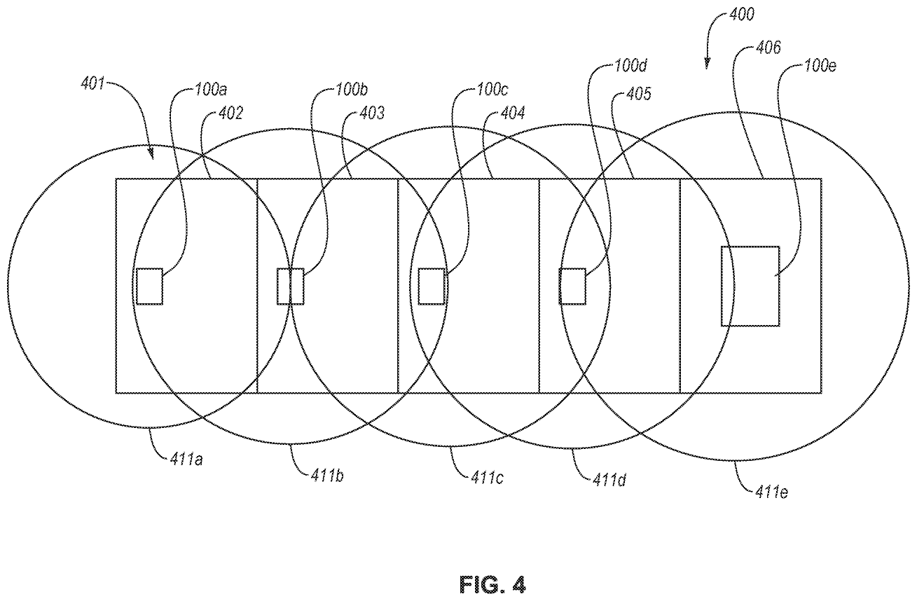

FIG. 4 is a system 400 for treating a plurality of enclosed spaces, according to an embodiment. The system 400 includes a plurality of room treatment devices 100a-100e in wireless communication with each other (e.g., through a daisy chain connection) throughout a plurality of rooms 402-406 of a building 401. In embodiments, the rooms 402-406 may be separate rooms in any of the buildings disclosed herein such as classrooms, hospital rooms, hotel rooms, court rooms, or the like. In some embodiments, the rooms 402-406 may be cruise-ship rooms, train cars, or the like. While five rooms 402-406 and corresponding room treatment devices 100a-100e are depicted the number of rooms and corresponding room treatment devices may be more or less than five, such as at least two, at least 5, two to 1000, five to 100, 20 to 50, 50 to 200, 100 to 500, less than 1000, less than 500, or less than 100 rooms and corresponding room treatment devices.

The plurality of room treatment devices 100a-100e of the system 400 may be similar or identical to the room treatment device 100 in one or more aspects. For example, each room treatment device of the plurality of room treatment devices 100a-100e includes a room treatment source, a controller configured to control treatment output from the room treatment source, and a wireless communication hub configured to receive and propagate one or more wireless signals from other or additional room treatment devices of the plurality of room treatment devices 100a-100e. For example, the first room treatment device 100a may be in direct communication with any of room treatment devices 100b-100e in an effective signal area 411a of the wireless communication hub of room treatment device 100a. The effective signal area 411a-411e of a specific room treatment device 100a-100e refers to the area in which the wireless communication hub corresponding thereto can wirelessly communicate (e.g., transmit and receive data) with wireless communication hubs of the other or additional room treatment devices located therein. The effective signal area may vary in size depending upon the type of wireless signal, such as Bluetooth, radio frequency, cellular, Wi-Fi, infrared, or the like. For example, the effective signal area of a room treatment device may be about 5 meters or more, such as 5 meters to 100 meters, 5 meters to 20 meters, 10 meters to 50 meters, 20 meters to 40 meters, less than 30 meters, or less than 20 meters.

As shown in FIG. 4, the first room treatment device 100a is in direct communication with second room treatment device 100b which is in an effective signal area 411a of the wireless communication hub of room treatment device 100a. The second room treatment device 100b is in direct communication with first room treatment device 100a and third room treatment device 100c which are in the effective signal area 411b of the wireless communication hub of room treatment device 100b. The third room treatment device 100c is in direct communication with second room treatment device 100b and fourth room treatment device 100d which are in the effective signal area 411c of the wireless communication hub of room treatment device 100c. The fourth room treatment device 100d is in direct communication with third room treatment device 100c and fifth room treatment device 100e which are in the effective signal area 411d of the wireless communication hub of room treatment device 100d. The fifth room treatment device 100e is in direct communication with fourth room treatment device 100d which is in the effective signal area 411e of the wireless communication hub of room treatment device 100e. While the fifth room treatment device 100e is not in direct communication with the first room treatment device 100a or within the effective signal area 411a thereof. However, by wirelessly communicating or otherwise linking to one or more room treatment devices therebetween, the fifth room treatment device 100e is indirectly connected to the first room treatment device 100a and may communicate wireless signals and data therewith. This daisy chain configuration even allows connections to wireless networks outside of the network formed between the room treatment devices when only one or some of the room treatment device are in the effective signal range or area of the wireless network outside of the plurality of room treatment devices. The connection to the outside network may be made when at least one of the plurality of room treatment devices is in an effective range of a Wi-Fi signal of an outside Wi-Fi network or connected to a LAN. In such examples, the system 400 may be in communication with the outside network, such as to transmit records, transmit alarm signals, transmit or receive room treatment data, or the like when one or more of the room treatment devices are not in an effective signal area (e.g., Wi-Fi signal area) of the outside network.

As shown from FIG. 4, multiple room treatment devices may be in the effective signal area of a single room treatment device, such as in adjacent rooms or in a floor above and/or below the floor on which the room treatment device sites. In such examples, the operational program includes instructions to propagate alarm signals only once, such as by identifying and confirming use of a unique alarm signal in a corresponding room treatment device.

In order to sanitize one or more of the plurality of rooms 402-406, the room treatment devices 100a-100e may include one or more of an ozone generator, an ion generator, an ultraviolet light source, a scent source, an anti-microbial source, a humidifier, a dehumidifier, or an air filter. In such embodiments, the room treatment devices 100a-100e may output a room treatment material (e.g., ozone, hydroxide ions, an anti-microbial material, UV light, or the like). The room treatment may be administered according to one or more operational programs stored in the memory of the controller of the room treatment device, such as one or more operational programs include machine readable and executable instructions for outputting one or more of ozone, hydroxide ions, hydride ions, a selected scent, ultraviolet light, or anti-microbial material according to a schedule. Such systems 400 may effectively treat (e.g., sanitize) an entire building or large portions thereof.

In embodiments where the room treatment devices 100a-100e include one or more of a microphone, a video camera, or a motion detector, the one or more operational programs may include instructions for ceasing treatment output if a sound is detected by the microphone or motion is detected by the motion detector or the video camera, such as ceasing treatment in a room if a sound is detected by the microphone or motion is detected by the motion detector or the video camera of a room treatment device in the room. The one or more operational programs may include instructions for recording one or more of sound via the microphone or video via the video camera. The one or more operational programs may include instructions for wirelessly transmitting one or more of the sound or video to a remote storage site, emergency services, or administrator.

The one or more operational programs may include machine readable and executable instructions for producing an alarm responsive to an alarm signal received by the wireless communication hub from an additional room treatment device of the plurality of room treatment devices, and propagating the alarm signal to any of the plurality of room treatment devices in communication with the wireless communication hub, responsive to the alarm signal.

The one or more operational programs may include machine readable and executable instructions for determining if the alarm signal is a unique signal such that multiple alarm signals are not propagated. A unique alarm signal identifier may be produced from the room treatment device where the alarm signal is initiated. Upon receiving the alarm signal with the unique alarm signal identifier from the first room treatment device, a second room treatment device may determine if the alarm signal with the unique alarm signal identifier has been received previously, such as from an additional or other room treatment device than the first room treatment device. If the second room treatment device has not previously received the signal from an additional room treatment device (and produced the alarm corresponding thereto), the operational program may include instructions to sound an alarm from the second room treatment device and propagate the alarm signal to additional room treatment devices in wireless communication therewith. If the second room treatment device has previously received the signal from another room treatment device and produced the alarm corresponding thereto, the second room treatment device may not sound an alarm or propagate the alarm signal further. Each room treatment device 100a-100e may include such instructions to prevent a repeating loop of alarm signals from propagating through the system 400.

While not depicted in FIG. 4, in some embodiments, at least one room treatment device of the plurality of room treatment devices 100a-100e may include a remote control in wireless communication with one or more of the controller or the wireless communication hub of the at least one room treatment device. The remote control is similar or identical to the remote control 190 (FIGS. 1 and 2). For example, the remote control may include one or more of buttons or a receiver configured to send one or more signals to the controller or wireless communication hub of the corresponding at least one room treatment device based on button pushes or verbal commands to actuate one or more functions of the at least one room treatment device. Accordingly, a remote control may be utilized to initiate or terminate room treatment, record events in the room, or initiate and alarm signal throughout the system 400. For example, the one or more operational programs include machine readable and executable instructions for initiating an alarm on the room treatment device and propagating an alarm signal to other room treatment devices of the plurality of room treatment devices via the wireless communication hub, responsive to input from the remote control or manual input on the room treatment device.

In some examples, the room treatment devices may have inputs that allow the user to selectively communicate with additional room devices of the plurality of room devices, such as by pressing a button or operating a switch.

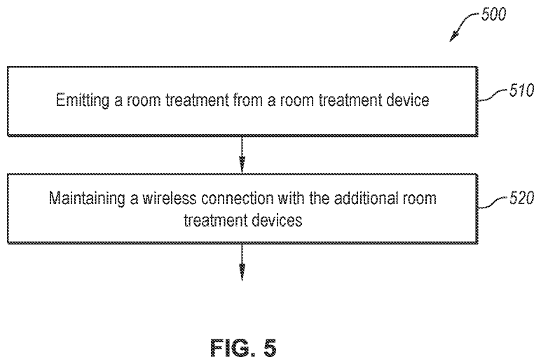

FIG. 5 is a flow diagram of a method 500 for treating a room, according to an embodiment. The method 500 includes a block 510 of emitting a room treatment from a room treatment device, the room treatment device including a room treatment source, a controller configured to control treatment output from the room treatment source, and a wireless communication hub configured to receive and propagate one or more wireless signals from additional room treatment devices, and a block 520 of maintaining a wireless connection with the additional room treatment devices. The method 500 may include more or fewer blocks than blocks 510 and 520. For example, blocks 510 and 520 may be combined or split into more blocks, block 510 may be omitted, or additional blocks may be added to blocks 510 and 520. In some examples, the order of the blocks 510 and 520 may be switched.

The block 510 of emitting a room treatment from a room treatment device may include using any of the room treatment devices disclosed herein, such as room treatment device 100. For example, the room treatment device may include a room treatment source, a controller configured to control treatment output from the room treatment source, and a wireless communication hub configured to receive and propagate one or more wireless signals from additional room treatment devices. Emitting a room treatment may include emitting the room treatment from the room treatment source. The room treatment device may include one or more sensing devices, one or more fans, a power source, a remote control, controls, or any other components disclosed herein.

The room treatment source may include one or more of an ozone generator, an ion generator, an ultraviolet light source, a scent source, an anti-microbial source, a humidifier, a dehumidifier, or an air filter. In such embodiments, emitting a room treatment from a room treatment device may include emitting one or more of ozone, hydroxide ions, hydride ions, a selected scent, ultraviolet light, water vapor, anti-microbial material, or any other material selected to sanitize objects in a room. Emitting a room treatment from a room treatment device may include generating the room treatment material (e.g., ozone, ions, a scented material, UV light) with the room treatment source. Emitting a room treatment from a room treatment device may include distributing the room treatment material with a fan, such as by blowing the room treatment material out of the room treatment device and into the room.

The controller of the room treatment device includes a memory storage medium storing one or more operational programs thereon, the one or more operational programs including machine readable and executable instructions for treatment output from the room treatment source, for connecting and maintaining wireless connections with additional room treatment devices, initiating and propagating alarm signals, locating a remote control, or any other functionality disclosed herein. The controller includes a processor operably coupled to the memory storage medium and configured to execute the one or more operational programs. The machine readable and executable instructions for treatment output from the room treatment source may include instructions for controlling one or more of duration, amount, time of, or type of treatment output from the treatment source. For example, emitting the room treatment may be carried out according to a schedule. The machine readable and executable instructions for treatment output from the room treatment source may include instructions for controlling one or more fans to selectively distribute the room treatment throughout the room.

Emitting a room treatment from a room treatment device may be responsive to an input from the controller or a remote control to initiate the room treatment output. Such inputs may include manual inputs such as button pushes, switches, dials or the like. The inputs may include voice commands spoken into a receiver of the room treatment device or remote control corresponding thereto. Accordingly, the method 500 may include inputting a command to initiate room treatment.

Emitting a room treatment from a room treatment device may include emitting a room treatment from a plurality of room treatment devices in a plurality of rooms. For example, the operational programs of a plurality of room treatment devices may include instructions for treating a room when the room is expected to be vacant (e.g., at night or prior to check-in for a hotel room).

The block 520 of maintaining a wireless connection with the additional room treatment devices may include maintaining a wireless connection with one or more additional room treatment devices of a plurality of room treatment devices, such as by one or more of a Bluetooth, Wi-Fi, cellular, infrared, or radio connection. The wireless connection may be made and maintained by the communication hub, such as using one or more antennas operably coupled thereto.

Maintaining a wireless connection with the additional room treatment devices may include making and maintaining direct wireless connections to one or more additional room treatment devices in an effective area of the wireless signal of the room treatment device. Maintaining a wireless connection with the additional room treatment devices may include making and maintaining indirect wireless connections to one or more additional room treatment devices outside of the effective area of the wireless signal of the room treatment device, such as via one or more additional room treatment devices therebetween (e.g., a daisy chain of room treatment devices. At least one of the plurality of room treatment devices may be outside of a signal area of an outside wireless network or not in communication with the outside wireless network. Accordingly, maintaining the wireless connection may include maintaining wireless connections to one or more additional room treatment devices may include maintaining (e.g., indirectly) a connection to an outside wireless network.

In some embodiments, the room the room treatment device includes one or more sensing devices, such as a microphone, a video camera, a speaker, or a motion detector. In such embodiments, the method 500 may further include ceasing treatment output if a sound is detected by the microphone or motion is detected by the motion detector or the video camera. Ceasing the treatment output may be carried out automatically by the controller according to an operational program having machine readable and executable instructions to deactivate the room treatment source if a sound is detected by the microphone or motion is detected by the motion detector or the video camera.

The method 500 may include recording one or more of sound via the microphone or video on the video camera. For example, the controller of the room treatment device may include operational programs having machine readable and executable instructions for recording one or more of sound via the microphone or video on the video camera responsive to an input (e.g., button push on the controls or remote control). For example, one or more operational programs may include instructions for recording one or more of sound via the microphone or video on the video camera, responsive to a manual input, remote input, sound detected above a threshold level, or identifying a firearm in images.

The method 500 may include wirelessly transmitting the one or more of the sound or video recording to another electronic storage device. For example, one or more operational programs may include instructions for wirelessly transmitting one or more of the sound or video to the computer of an administrator, emergency services, or a remote storage site (e.g., server). One or more operational programs may include instructions for wirelessly transmitting one or more sound or video recordings from an additional room treatment device to the computer of an administrator, emergency services, or a remote storage site. The recordings may be finally transmitted to another electronic storage device through an outside network, such as a Wi-Fi network or LAN, not connected to at least some of the plurality of room treatment devices.

The recordings may be supplied to or processed with an image processing software to identify individuals or objects present. For example, the recorded images may be processed to identify the presence of prohibited individuals or objects of concern (e.g., gun, fire, or the like) responsive thereto, an operational program may instructed the initiation of an alarm signal.

The method 500 may include initiating an alarm on the plurality of room treatment devices. Initiating the alarm may include inputting an alarm initiation signal into the controls of the fluid collection device or the remote control corresponding therewith. The inputs may be a single button push, a selected sequence of button pushes, a voice input to activate an alarm, or any other input to initiate an alarm disclosed herein. For example, the one or more operational programs may include instructions for initiating an alarm and providing an alarm signal to additional room treatment devices in communication therewith responsive to a manual input, remote input, or sound detected above a threshold level, or identifying a firearm in images captured on the video camera.

The method 500 may include producing an alarm responsive to an alarm signal received by the wireless communication hub from one of the additional room treatment devices. For example, producing the alarm may include producing an audible alarm from a speaker on the room treatment device. The method 500 may include propagating the alarm signal to one or more of the additional room treatment devices in communication with the wireless communication hub, responsive to the alarm signal. Propagating the alarm signal to one or more of the additional room treatment devices may include propagating the alarm signal via the wireless communication hub of the room treatment device.

The method 500 may include determining if the alarm signal indicates an alarm should be emitted from the room treatment device (e.g., through the speaker thereon) or the alarm signal should be propagated to additional room treatment devices. For example, the method 500 may include determining if the alarm signal is a unique alarm signal. In such embodiments, the operational programs may include machine readable and executable instructions for determining if the alarm signal is a unique signal such that a single alarm signal is not propagated throughout a plurality of room treatment devices multiple times. For example, a unique alarm signal identifier may be produced from the room treatment device where the alarm signal is initiated. Upon receiving the alarm signal with the unique alarm signal identifier from an initial room treatment device, the room treatment device may determine if the alarm signal with the unique alarm signal identifier has been received from any other room treatment device than the initial room treatment device. If the room treatment device has not previously received the alarm signal from another room treatment device, the instructions direct the room treatment device to sound an alarm and propagate the alarm signal to additional room treatment devices in wireless communication therewith. If the room treatment device has previously received the signal from another room treatment device, the instructions direct the room treatment device not to sound an alarm or propagate the alarm signal further.

In some embodiments, at least one of the room treatment device or additional room treatment devices may be operably coupled to an outside network (e.g., a wireless network outside of the room treatment device or additional room treatment devices), via the wireless communication hub thereof. For example, the outside wireless network may include a Wi-Fi network or LAN in a building. In such embodiments, the wireless communication between the room treatment devices may be operated in isolation from the outside wireless network until a command to communicate with the outside network is executed, such as instructions to transmit an alarm or recording to an administrator, emergency services, a storage device, or the like. The method 500 may include communicating the alarm signal to a device on the outside wireless network, such as a computer of an administrator, emergency services, or storage site (e.g., servers).

One or more of the room treatment devices may include a remote control in wireless communication with one or more of the controller or the wireless communication hub of the at least one room treatment device. The remote control may include one or more of buttons or a receiver configured to send one or more signals to the controller or wireless communication hub based on button pushes or verbal commands to actuate one or more functions of the at least one room treatment device. In such embodiments, the method 500 may include initiating an alarm on the room treatment device and propagating the alarm signal to other room treatment devices of the plurality of room treatment devices via the wireless communication hub, such as responsive to input from the remote control or manual input on the room treatment device. For example, the method 500 may include pressing an alarm button, inputting a selected sequence of button pushes, speaking a code to initiate an alarm into a microphone on the remote control, or any other input.

The method 500 may include inputting commands to adjust or terminate room treatment, such as manually or by voice commands. In embodiments utilizing the remote control, the method 500 may include pressing a button, inputting a selected sequence of button pushes, or speaking a code into the remote control to initiate a recording, terminate a recording, initiate room treatment, adjust a parameter (e.g., type, duration, amount) of a room treatment, or terminate a room treatment.

The method 500 may include utilizing any of the functionalities or the room treatment devices and systems disclosed herein.

The embodiments disclosed herein provide for both sanitization of spaces and communication of safety concerns throughout multiple rooms, without the necessity of having a single outside network connected to all of the room treatment devices. The operation(s) of the room treatment devices in separate rooms may be controlled in unison or separately, while an emergency situation may be communicated to all room treatment devices substantially simultaneously, even in the absence of an outside network.

As used herein, the term “about” or “substantially” refers to an allowable variance of the term modified by “about” by ±10% or ±5%. Further, the terms “less than,” “or less,” “greater than”, “more than,” or “or more” include as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting. Additionally, the words “including,” “having,” and variants thereof (e.g., “includes” and “has”) as used herein, including the claims, shall be open ended and have the same meaning as the word “comprising” and variants thereof (e.g., “comprise” and “comprises”).

What is claimed is:

1. A room treatment device having networking capabilities, the room treatment device comprising:
a room treatment source;
a controller configured to control treatment output from the room treatment source; and
a wireless communication hub configured to receive and propagate one or more wireless signals from other room treatment devices.

2. The room treatment device of claim 1 wherein room treatment source includes one or more of an ozone generator, an ion generator, an ultraviolet light source, a scent source, an anti-microbial source, a humidifier, a dehumidifier, or an air filter.

3. The room treatment device of claim 1 wherein the controller includes:
a memory storage medium storing one or more operational programs thereon, the one or more operational programs including machine readable and executable instructions for treatment output from the room treatment source; and
a processor operably coupled to the memory storage medium and configured to execute the one or more operational programs.

4. The room treatment device of claim 3 wherein the one or more operational programs include machine readable and executable instructions for outputting one or more of ozone, hydroxide ions, hydride ions, a selected scent, ultraviolet light, or anti-microbial material according to a schedule.

5. The room treatment device of claim 3 further comprising one or more of a microphone, a video camera, or a motion detector.

6. The room treatment device of claim 5 wherein the one or more operational programs include instructions for ceasing treatment output if a sound is detected by the microphone or motion is detected by the motion detector or the video camera.

7. The room treatment device of claim 5 wherein the one or more operational programs include instructions for recording one or more of sound via the microphone or video on the video camera.

8. The room treatment device of claim 7 wherein the one or more operational programs include instructions for wirelessly transmitting the one or more of the sound or video to a remote storage site.

9. The room treatment device of claim 3 wherein the one or more operational programs include machine readable and executable instructions for:
producing an alarm responsive to an alarm signal received by the wireless communication hub from an additional room treatment device; and
propagating the alarm signal to all additional room treatment devices in communication with the wireless communication hub responsive to the alarm signal.

10. The room treatment device of claim 1 further comprising a remote control in wireless communication with one or more of the controller or the wireless communication hub, the remote control including one or more of buttons or a receiver configured to send one or more signals to the controller based on button pushes or verbal commands to actuate one or more functions of the room treatment device.

11. The room treatment device of claim 3 wherein the one or more operational programs include machine readable and executable instructions for initiating an alarm and propagating an alarm signal via the wireless communication hub responsive to input from the remote control or manual input on the room treatment device.

12. The room treatment device of claim 1 wherein the wireless signal includes one or more of Bluetooth, Wi-Fi, cellular, infrared, or radio signals.

13. A system for treating a plurality of enclosed spaces, the system comprising:
a plurality of room treatment devices, each room treatment device of the plurality of room treatment devices including:
a room treatment source;
a controller configured to control treatment output from the room treatment source; and
a wireless communication hub configured to receive and propagate one or more wireless signals from other room treatment devices of the plurality of room treatment devices.

14. The system of claim 13 wherein room treatment source includes one or more of an ozone generator, an ion generator, an ultraviolet light source, a scent source, an anti-microbial source, a humidifier, a dehumidifier, or an air filter.

15. The system of claim 13 wherein the controller includes:
a memory storage medium storing one or more operational programs thereon, the one or more operational programs including machine readable and executable instructions for treatment output from the room treatment source; and
a processor operably coupled to the memory storage medium and configured to execute the one or more operational programs.

16. The system of claim 15 wherein the one or more operational programs include machine readable and executable instructions for outputting one or more of ozone, hydroxide ions, hydride ions, a selected scent, ultraviolet light, or anti-microbial material according to a schedule.

17. The system of claim 15 further comprising one or more of a microphone, a video camera, or a motion detector.

18. The system of claim 17 wherein the one or more operational programs include instructions for ceasing treatment output if a sound is detected by the microphone or motion is detected by the motion detector or the video camera.

19. The system of claim 17 wherein the one or more operational programs include instructions for recording one or more of sound via the microphone or video on the video camera.

20. The system of claim 19 wherein the one or more operational programs include instructions for wirelessly transmitting the one or more of the sound or video to a remote storage site.

21. The system of claim 15 wherein the one or more operational programs include machine readable and executable instructions for:
producing an alarm responsive to an alarm signal received by the wireless communication hub from an additional room treatment device of the plurality of room treatment devices; and
propagating the alarm signal to any of the plurality of room treatment devices in communication with the wireless communication hub, responsive to the alarm signal.

22. The system of claim 15 wherein at least one room treatment device of the plurality of room treatment devices includes a remote control in wireless communication with one or more of the controller or the wireless communication hub of the at least one room treatment device, the remote control including one or more of buttons or a receiver configured to send one or more signals to the controller or wireless communication hub based on button pushes or verbal commands to actuate one or more functions of the at least one room treatment device.

23. The system of claim 22 wherein the one or more operational programs include machine readable and executable instructions for initiating an alarm on the room treatment device and propagating an alarm signal to other room treatment devices of the plurality of room treatment devices via the wireless communication hub responsive to input from the remote control or manual input on the room treatment device.

24. The system of claim 13 wherein the wireless signal includes one or more of Bluetooth, Wi-Fi, cellular, infrared, or radio signals.

25. The system of claim 13 wherein at least one of the plurality of room treatment devices is operably coupled to a wireless network outside of the plurality of room treatment devices, via the wireless communication hub.

26. The system of claim 25 wherein at least one of the plurality of room treatment devices is outside of a signal area of the wireless network or not in communication with the wireless network.

27. A method for treating a room, the method comprising:
emitting a room treatment from a room treatment device, the room treatment device including:
a room treatment source;
a controller configured to control treatment output from the room treatment source; and
a wireless communication hub to receive and propagate one or more wireless signals from additional room treatment devices; and
maintaining a wireless connection with the additional room treatment devices.

28. The method of claim 27 wherein emitting a room treatment from a room treatment device includes emitting one or more of ozone, hydroxide ions, hydride ions, a selected scent, ultraviolet light, or anti-microbial material.

29. The method of claim 27 wherein the controller includes:
a memory storage medium storing one or more operational programs thereon, the one or more operational programs including machine readable and executable instructions for treatment output from the room treatment source; and
a processor operably coupled to the memory storage medium and configured to execute the one or more operational programs.

30. The method of claim 27 wherein emitting the room treatment is carried out according to a schedule.

31. The method of claim 27 wherein the room the room treatment device includes one or more of a microphone, a video camera, or a motion detector.

32. The method of claim 31 further comprising ceasing treatment output if a sound is detected by the microphone or motion is detected by the motion detector or the video camera.

33. The method of claim 31 further comprising recording one or more of sound via the microphone or video on the video camera.

34. The method of claim 33 further comprising wirelessly transmitting the one or more of the sound or video to a remote storage site.

35. The method of claim 27 further comprising:
producing an alarm responsive to an alarm signal received by the wireless communication hub from one of the additional room treatment devices; and
propagating the alarm signal to one or more of the additional room treatment devices in communication with the wireless communication hub, responsive to the alarm signal.

36. The method of claim 35 wherein at least one of the room treatment device or additional room treatment devices is operably coupled to a wireless network outside of the room treatment device or additional room treatment devices, via the wireless communication hub thereof.

37. The method of claim 36 further comprising communicating the alarm signal to a device on the wireless network outside of the room treatment device or additional room treatment devices.

38. The method of claim 27 wherein the room treatment device includes a remote control in wireless communication with one or more of the controller or the wireless communication hub of the room treatment device, the remote control including one or more of buttons or a receiver configured to send one or more signals to the controller or wireless communication hub based on button pushes or verbal commands to actuate one or more functions of the room treatment device.

39. The method of claim 38 further comprising initiating an alarm on the room treatment device and propagating an alarm signal to additional treatment devices via the wireless communication hub responsive to input from the remote control or manual input on the room treatment device.
